(19) United States
(12) Patent Application Publication
(10) Pub. No.: US 2010/0035882 A1
(43) Pub. Date: Feb. 11, 2010

(54) INHIBITION OF PDE2A

(76) Inventors: Peter Ellinghaus, Wuppertal (DE); Andreas Wilmen, Koln (DE); Martin Hendrix, Odenthal (DE); Adrian Tersteegen, Wuppertal (DE)

Correspondence Address: EICHEN & ASSOCIATES, 44 EAST COURT STREET, DOYLESTOWN, PA, 18901, US

(21) Appl. No.: 12/444,708

(22) PCT Filed: Oct. 2, 2007
(86) PCT No.: PCT/EP2007/008561

§ 371 Date: Apr. 8, 2009

(30) Foreign Application Priority Data
Oct. 14, 2006 (DE) 10 2006 048 693.5

(51) Int. Cl. A61K 31/53; A61P 9/00

(52) U.S. Cl. 514/243

(57) ABSTRACT

The invention relates to the use of PDE2A inhibitors for the manufacture of a medicament for the treatment and/or prophylaxis of coronary diseases, especially stable and unstable angina pectoris, acute myocardial infarction, prophylaxis of myocardial infarction, heart failure, and high blood pressure and the sequelae of atherosclerosis, and vascular disorders, disorders of the kidney, especially renal failure, inflammatory disorders, erectile dysfunction and prevention of sudden heart death.

The invention relates to the use of PDE2A inhibitors for the manufacture of a medicament for the treatment and/or prophylaxis of cardiac disorders, especially of heart failure and its underlying cardiomyopathies such as dilated cardiomyopathy (DCM), restrictive cardiomyopathy (RCM), arrhythmogenic right-ventricular cardiomyopathy (ARVCM), myocarditis and especially hypertrophic cardiomyopathy (HCM). The invention additionally relates to the treatment of erectile impairments, high blood pressure and the prevention of arteriosclerosis using PDE2A inhibitors.

Essential hypertension damages not only the brain, kidneys and vessels but also in particular the heart. Before the introduction of antihypertensive therapy, essential hypertension was the main cause of heart failure and myocardial infarction. Even now the risk of developing heart failure and suffering a myocardial infarction is greater for a hypertensive person even if receiving antihypertensive treatment than for a normotensive person. Left ventricular hypertrophy is the principle structural mechanism for adapting the myocardium to the chronic pressure-load during essential hypertension. The extent of the myocardial hypertrophy increases with the level of the blood pressure.

In the early stage of essential hypertension, the systolic wall tension, the left ventricular afterload—is raised as a consequence of the systolic pressure load applied to the ventricular wall. The left heart hypertrophy which develops results in the wall tension being normalized again as a consequence of an increase in thickness of the myocardial walls. In this early stage of concentric left heart hypertrophy, the left ventricle is able to deliver a normal cardiac output with a normal cardiac energy consumption per unit weight of myocardium, despite hypertensive systolic blood pressures. Even in this stage there is an impairment of the diastolic function of the left ventricle.

A chronic pressure load on the left ventricle leads to an abnormal activation of fetal growth factors which alter protein biosynthesis and fetal muscle gene products. Angiotensin II, noradrenaline and other growth hormones have a growth-promoting effect on the myocardium. This effect results, irrespective of the systolic pressure load, in a further stimulation of the development of myocardial hypertrophy. Depending on the extent of the growth hormones, it is possible in some circumstances for there to be a development of a myocardial hypertrophy which is disproportionately large for the increase in blood pressure and is similar to a hypertrophic cardiomyopathy. As a consequence of the fact that the adult myocardial tissue has little or no capability for cell division, owing to blockade of the cell cycle, the result on the molecular level is a pathological hypertrophic reaction of the myocardium. Cardiac hypertrophy is not a physiological mechanism for adaptation to the chronic continuous load. It is an independent risk factor for cardiac events such as myocardial infarction, heart failure and sudden heart death [1].

Therapeutic methods and active ingredients which prevent cardiac hypertrophy are thus suitable for treating symptoms of the abovementioned disorders.

The DNA sequence which codes for human PDE2A is shown in SEQ ID NO: 1 in the sequence listing. The amino acid sequence of human PDE2A is shown in SEQ ID NO: 2 of the sequence listing.

As shown in FIG. 1, it has surprisingly been observed in a cellular hypertrophy model that expression of PDE2A-mRNA is increased on the induction of the hypertrophy. For this purpose, the rat cardiomyocyte cell line H9c2 (ATCC number: CRL-1446) was exposed to a hypertrophic stimulus by arginine-vasopressin which is expressed inter alia in an increased expressed of marker genes for cardiac hypertrophy such as ANP (atrial natriuretic peptide) and MYHCB (myosin heavy chain beta-subunit) [2]. An increased expression of the cGMP-hydrolyzing PDE2A is able to reduce the intracellular cGMP level of the cardiomyocytes and thus suppress the antihypertrophic effect of cGMP [3,4]. It can be inferred from this observation that the increased expression of PDE2A in hypertrophic H9c2 cells also contributes in vivo to the pathogenesis of cardiac hypertrophy, and an inhibition of the PDE2A activity by a small molecule drug has a positive effect on cardiac hypertrophy, because the cGMP level in the cardiomyocytes remains high and thus the antihypertrophic effect of cGMP is maintained [5]. To examine this hypothesis, H9c2 cells were stimulated with arginine-vasopressin and incubated with the PDE2 inhibitor BAY 60-7550 [6]. BAY 60-7550 is the substance 2-(3,4-dimethoxybenzyl)-7-[1-(1-hydroxyethyl)-4-phenylbutyl]-5-methylimidazo[5,1-f][1,2,4]triazin-4(3H)-one having the structural formula:

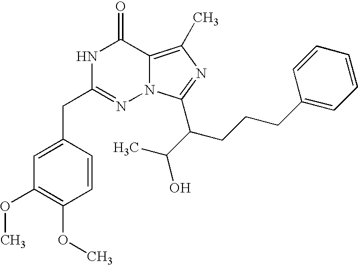

As shown in FIG. 2, the PDE2A inhibitor BAY 60-7550 is capable of dose-dependent suppression of the increase in the hypertrophy marker gene MYHCB. In order to verify that incubation of H9c2 cells with the PDE2A inhibitor BAY 60-7550 also leads to an increase in the intracellular cGMP level and thus has an antihypertrophic effect, the intracellular cGMP content was determined by EIA after stimulation of cGMP synthesis by ANP in the presence of the PDE2A inhibitor BAY 60-7550. As is evident from FIG. 3, BAY 60-7550 dose-dependently increases the intracellular cGMP content in H9c2 cells. To verify the in vitro findings, the antihypertrophic effect of the PDE2 inhibitor BAY 60-7550 was investigated in vivo in a mouse hypertrophy model. For this purpose, mice of the C57BL6 strain received subcutaneous administration of 2 mg/kg isoprenaline once a day and, as positive control, 10 mg/kg enalapril administered via the drinking water in addition to the isoprenaline. BAY 60-7550 was administered in parallel with the isoprenaline injection 2× a day with a dose of 10 mg/kg i.p. As is evident from FIG. 4, infusion of isoprenaline increases the weight of the animal's heart in relation to the body weight. Just like the positive control enalapril, administration of the PDE2A inhibitor BAY 60-7550 led in both dose groups to a marked reduction in the ratio of the weight of the heart to the body weight.

It is evident from the data that PDE2A inhibition could also prevent cardiac hypertrophy in humans.

The present invention therefore relates to the use PDE2A inhibitors for the manufacture of a medicament for the treatment and/or prophylaxis of the following diseases: coronary heart diseases, especially stable and unstable angina pectoris, acute myocardial infarction, myocardial infarction prophylaxis, sudden heart death, heart failure, and high blood pressure and the sequelae of atherosclerosis, and vascular disorders, kidney disorders, and erectile dysfunction.

Antagonists in the sense of the invention are all substances which bring about an inhibition of the biological activity of PDE2A. Particularly preferred antagonists are nucleic acids including locked nucleic acids, peptide nucleic acids and “spiegelmers”, proteins including antibodies and low molecular weight substances; very particularly preferred antagonists are low molecular weight substances.

The invention relates to:
1. The use of a PDE2A polypeptide or of a nucleic acid which encodes a PDE2A polypeptide in an assay system for finding inhibitors of PDE2A suitable for the treatment and/or prophylaxis of heart failure and of the cardiomyopathies underlying it.
   A nucleic acid encoding a PDE2A polypeptide is a nucleic acid selected from the group consisting of:
   a) nucleic acid molecules which encode a polypeptide which includes the amino acid sequence disclosed by SEQ ID NO: 2, and functional fragments thereof,
   b) nucleic acid molecules which include the sequence depicted in SEQ ID NO: 1, and functional fragments thereof,
   c) nucleic acid molecules whose complementary strand hybridizes with a nucleic acid molecule from a) or b) under stringent conditions and which have the biological function of a PDE2A, a stringent hybridization of nucleic acid molecules being carried out in an aqueous solution which comprises 0.2×SSC (1×standard saline-citrate=150 mM NaCl, 15 mM trisodium citrate) at 68° C. (Sambrook et al., 1989); and
   d) nucleic acid molecules which differ by reason of the degeneracy of the genetic code from those mentioned under c).

A PDE2A polypeptide in the sense of the invention is a polypeptide which is encoded by one of the nucleic acids mentioned under a)-d). A polypeptide is in particular one including the sequence depicted in SEQ ID NO: 1 or including a fragment thereof which has PDE2A activity, a PDE2A polypeptide.
2. The use as set forth in item 1, where the assay system is cell-free.
3. The use as set forth in item 1, where whole cells which comprise a nucleic acid which encodes a PDE2A are used in the assay system. It is possible in this connection for the nucleic acid to have been endogenously present or introduced recombinantly.
4. The use as set forth in items 1-3, where a PDE2A activity is measured.
5. The use as set forth in item 4, where the cGMP or the GMP level is measured.
6. The use as set forth in items 1-3, where expression of PDE2A is measured.
7. The use as set forth in items 1-6, where the heart failure is a heart failure induced by a cardiomyopathy selected from the group of cardiomyopathies consisting of dilated cardiomyopathy (DCM), restrictive cardiomyopathy (RCM), arrhythmogenic right-ventricular cardiomyopathy (ARVCM), myocarditis and/or hypertrophic cardiomyopathy (HCM).
8. The use of a PDE2A inhibitor which has been identified by means of one of the methods set forth in items 1-7 for the manufacture of a medicament for the treatment and/or prophylaxis of heart failure.
9. The use of a PDE2A inhibitor which has been identified by means of one of the methods set forth in items 1-7 for the manufacture of a medicament for the treatment and/or prophylaxis of heart failure induced by a cardiomyopathy selected from the group of cardiomyopathies consisting of dilated cardiomyopathy (DCM), restrictive cardiomyopathy (RCM), arrhythmogenic right-ventricular cardiomyopathy (ARVCM), myocarditis and/or hypertrophic cardiomyopathy (HCM).
10. The use of a PDE2A-specific antibody, of a PDE2A-specific antisense oligonucleotide or of a PDE2A-specific siRNA for the manufacture of a medicament for the treatment and/or prophylaxis of heart failure.
11. The use of a PDE2A-specific antibody, of a PDE2A-specific antisense oligonucleotide or of a PDE2A-specific siRNA for the manufacture of a medicament for the treatment and/or prophylaxis of heart failure induced by a cardiomyopathy selected from the group of cardiomyopathies consisting of dilated cardiomyopathy (DCM), restrictive cardiomyopathy (RCM), arrhythmogenic right-ventricular cardiomyopathy (ARVCM), myocarditis and/or hypertrophic cardiomyopathy (HCM). An siRNA is a short interfering RNA. Methods for providing PDE2A-specific antisense oligonucleotides, antibodies or siRNAs are known to the skilled worker. Suitable antisense oligonucleotides, siRNAs or antibodies eventually lead to inhibition of PDE2A activity. This may take place by a mechanism directly involving the PDE2A protein, or else acts at the level of transcription or translation of PDE2A.
12. The use of PDE2A inhibitor for the manufacture of a medicament for the treatment and/or prophylaxis of heart failure.
13. The use as set forth in item 12, where the heart failure is a heart failure induced by a cardiomyopathy selected from the group of cardiomyopathies consisting of dilated cardiomyopathy (DCM), restrictive cardiomyopathy (RCM), arrhythmogenic right-ventricular cardiomyopathy (ARVCM), myocarditis and/or hypertrophic cardiomyopathy (HCM).
14. The use according to items 12 or 13, where the PDE2A inhibitor has an IC₅₀ of less than 1 μM.
15. The use according to items 12 or 13, where the PDE2A inhibitor has an IC₅₀ of less than 100 nM.

The PDE2A inhibition can be measured for example in the PDE2A inhibition assay described below.

The PDE2A antagonists preferred in this connection show an inhibition in the PDE2A inhibition assay indicated below with an IC₅₀ of 1 μM, preferably with an IC₅₀ of less than 0.1 RIM.

The PDE2A inhibitors of the invention preferably cannot cross the blood/brain barrier and have systemic and not central effects.

The present invention also relates to the use of compounds of the general formula (I),

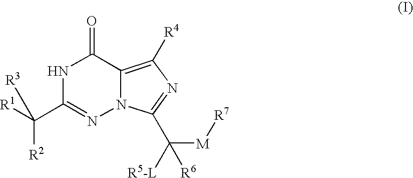

in which
R¹ is phenyl, naphthyl, quinolinyl or isoquinolinyl, each of which may be substituted up to three times, identically or differently, by radicals selected from the group consisting of (C₁-C₄)-alkyl, (C₁-C₄)-alkoxy, halogen, cyano, —NHCOR⁸, —NHSO₂R⁹, —SO₂NR¹⁰R¹¹, —SO₂R¹², and —NR¹³R¹⁴,
   in which
   R⁸, R¹⁰, R¹¹, R¹³ and R¹⁴ are independently of one another hydrogen or (C₁-C₄)-alkyl, and
   R⁹ and R¹² are independently of one another (C₁-C₄)-alkyl,
   or
   R¹⁰ and R¹¹ together with the adjacent nitrogen atom form an azetidin-1-yl, pyrrol-1-yl, piperid-1-yl, azepin-1-yl, 4-methylpiperazin-1-yl or morpholin-1-yl radical,
   or
   R¹³ and R¹⁴ together with the adjacent nitrogen atom form an azetidin-1-yl, pyrrol-1-yl, piperid-1-yl, azepin-1-yl, 4-methylpiperazin-1-yl or morpholin-1-yl radical,
R² and R³ are independently of one another hydrogen or fluorine,
R⁴ is (C₁-C₄)-alkyl,
R⁵ is (C₁-C₃)-alkyl,
R⁶ is hydrogen or methyl,
R⁷ is phenyl, thiophenyl, furanyl, each of which may be substituted up to three times identically or differently by radicals selected from the group consisting of (C₁-C₄)-alkyl, (C₁-C₄)-alkoxy, halogen and cyano, or is (C₅-C₈)-cycloalkyl,
L is carbonyl or hydroxymethanediyl, and
M is (C₂-C₅)-alkanediyl, (C₂-C₅)-alkenediyl or (C₂-C₅)-alkynediyl,
and the physiologically tolerated salts for the manufacture of a medicament for the treatment and/or prophylaxis of heart failure.

(C₁-C₄)-Alkyl and (C₁-C₃)-alkyl are in the context of the invention a straight-chain or branched alkyl radical having respectively 1 to 4 and 1 to 3 carbon atoms. Examples which may be mentioned are: methyl, ethyl, n-propyl, isopropyl, i-, s-, t-butyl. Methyl and ethyl are preferred.

(C₂-C₅)-Alkanediyl is in the context of the invention a straight-chain or branched alkanediyl radical having 2 to 5 carbon atoms. Examples which may be mentioned are ethylene, propane-1,3-diyl, propane-1,2-diyl, propane-2,2-diyl, butane-1,3-diyl, butane-2,4-diyl, pentane-2,4-diyl. A straight-chain (C₂-C₅)-alkane-1,ω-diyl radical is preferred. Examples which may be mentioned are ethylene, propane-1,3-diyl, butane-1,4-diyl, pentane-1,5-diyl. Propane-1,3-diyl and butane-1,4-diyl are particularly preferred.

(C₂-C₅)-Alkenediyl is in the context of the invention a straight-chain or branched alkenediyl radical having 2 to 5 carbon atoms. Examples which may be mentioned are ethene-1,2-diyl, ethene-1,1-diyl, propene-1,1-diyl, propene-1,2-diyl, prop-2-ene-1,3-diyl, propene-3,3-diyl, propene-2,3-diyl, but-2-ene-1,4-diyl, pent-2-ene-1,4-diyl. A straight-chain (C₂-C₅)-alkene-1,ω-diyl radical is preferred. Examples which may be mentioned are ethene-1,2-diyl, prop-2-ene-1,3-diyl, but-2-ene-1,4-diyl, but-3-ene-1,4-diyl, pent-2-ene-1,5-diyl, pent-4-ene-1,5-diyl. Prop-2-ene-1,3-diyl, but-2-ene-1,4-diyl and but-3-ene-1,4-diyl are particularly preferred.

(C₂-C₅)-Alkynediyl is in the context of the invention a straight-chain or branched alkynediyl radical having 2 to 5 carbon atoms. Examples which may be mentioned are ethyne-1,2-diyl, ethyne-1,1-diyl, prop-2-yne-1,3-diyl, prop-2-ynen-1,1-diyl, but-2-yne-1,4-diyl, pent-2-yne-1,4-diyl. A straight-chain (C₂-C₅)-alkene-1,ω-diyl radical is preferred. Examples which may be mentioned are ethyne-1,2-diyl, prop-2-yne-1,3-diyl, but-2-yne-1,4-diyl, but-3-yne-1,4-diyl, pent-2-yne-1,5-diyl, pent-4-yne-1,5-diyl. Prop-2-yne-1,3-diyl, but-2-yne-1,4-diyl and but-3-yne-1,4-diyl are particularly preferred.

(C₁-C₄)-Alkoxy is in the context of the invention a straight-chain or branched alkoxy radical having 1 to 4 carbon atoms. Examples which may be mentioned are: methoxy, ethoxy, n-propoxy, isopropoxy, t-butoxy, n-pentoxy and n-hexoxy. Methoxy and ethoxy are particularly preferred.

(C₅-C₈)-Cycloalkyl is in the context of the invention cyclopentyl, cyclohexyl, cycloheptyl or cyclooctyl. Those which may be preferably mentioned are: cyclopentyl, cyclohexyl or cycloheptyl.

Halogen is in the context of the invention generally fluorine, chlorine, bromine and iodine. Fluorine, chlorine and bromine are preferred. Fluorine and chlorine are particularly preferred.

Salts preferred in the context of the invention are physiologically acceptable salts of the compounds of the invention.

Physiologically acceptable salts of the compounds of the invention may be acid addition salts of the substances of the invention with mineral acids, carboxylic acids or sulfonic acids. Particular preferred examples are salts with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid.

However, salts which may also be mentioned are salts with conventional bases such as, for example alkali metal salts (e.g. sodium or potassium salts), alkaline earth metal salts (e.g. calcium or magnesium salts) or ammonium salts derived from ammonia or organic amines such as, for example, diethylamine, triethylamine, ethyldiisopropylamine, procaine, dibenzylamine, N-methylmorpholine, dihydroabietylamine, 1-ephenamine or methylpiperidine.

The compounds of the invention may exist in stereoisomeric forms which are related either as image and mirror image (enantiomers) or which are not related as image and mirror image (diastereomers). The invention relates both to the enantiomers or diastereomers or to the mixtures thereof in each case. The racemic forms can, just like the diastereomers, be separated into the stereoisomerically pure constituents in a known manner.

The use of compounds of the general formula (I) where R¹ is phenyl whose meta and/or para positions are substituted up to three times identically or differently by radicals selected from the group consisting of (C₁-C₄)-alkyl, (C₁-C₄)-alkoxy and —SO₂NR¹⁰R¹¹, and R², R³, R⁴, R⁵, R⁶, R⁷, R¹⁰, R¹¹, L and M have the meaning indicated above, for the manufacture of a medicament for the treatment and/or prophylaxis of heart failure is preferred.

The meta and para positions of the phenyl ring mean those positions which are respectively meta and para in relation to the CR²R³ group. These positions can be illustrated by the following structural formula (Ic):

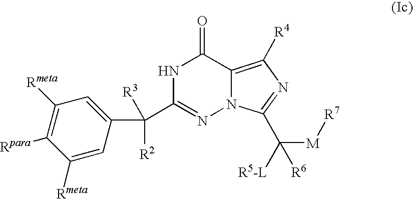

The use of compounds of the general formula (Ic) in which the para position and one meta position of the phenyl radical, are substituted, and the second meta position is unsubstituted, for the manufacture of a medicament for the treatment and/or prophylaxis of heart failure is particularly preferred.

It is likewise preferred to use compounds of the general formula (I), where R⁷ is phenyl, and R¹, R², R³, R⁴, R⁵, R⁶, L and M have the meaning indicated above, for the manufacture of a medicament for the treatment and/or prophylaxis of heart failure.

It is very particularly preferred to use compounds of the general formula (I),

where
R¹ is phenyl whose meta and/or para positions are substituted up to three times identically or differently by radicals selected from the group consisting of (C₁-C₄)-alkyl, (C₁-C₄)-alkoxy and —SO₂NR¹⁰R¹¹, or naphthyl or quinolinyl,
   in which R¹⁰ and R¹¹ are independently of one another hydrogen or (C₁-C₄)-alkyl,
R¹ and R² are hydrogen,
R⁴ is methyl or ethyl,
R⁵ is methyl,
R⁶ is hydrogen or methyl,
L is carbonyl or hydroxymethanediyl, and
M is straight-chain (C₂-C₅)-alkane-1,ω-diyl, straight-chain (C₂-C₅)-alkene-1,ω-diyl or straight-chain (C₂-C₅)-alkyne-1,ω-diyl, for the manufacture of a medicament for the treatment and/or prophylaxis of heart failure.

It is likewise preferred to use the substance 2-(3,4-dimethoxybenzyl)-7-[1-(1-hydroxyethyl)-4-phenylbutyl]-5-methylimidazo[5,1-f][1,2,4]triazin-4(3H)-one having the structural formula:

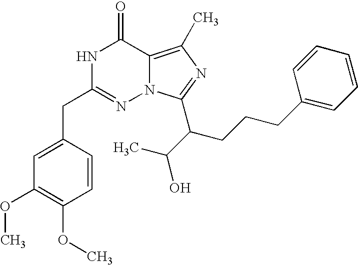

for the manufacture of a medicament for the treatment and/or prophylaxis of heart failure.

The use described above of the structural formulae disclosed above is also preferred where the heart failure is a heart failure induced by a cardiomyopathy selected from the group of cardiomyopathies consisting of dilated cardiomyopathy (DCM), restrictive cardiomyopathy (RCM), arrhythmogenic right-ventricular cardiomyopathy (ARVCM), myocarditis and/or hypertrophic cardiomyopathy (HCM).

The compound described above, its effect as PDE2 inhibitors, and processes for the preparation thereof are disclosed in WO 02/050078 A1.

### DESCRIPTION OF THE FIGURES

FIG. 1: Comparison of the relative expression of PDE2A-RNA in H9c2 cells after incubation with 1 micromole of arginine-vasopressin for 72 h. The expression of PDE2A relative to L32 ribosomal protein (rE) in H9c2 rat cardiomyocytes is shown. The results are shown in a table (mean relative expression rE from a triplicate determination. Mean Ct values for PDE2A and L32).

| | Ct | | | | |
|---|---|---|---|---|---|
| | r-PDE2A | Ct r-L32 | rE | MW | STABW |
| Control | 29.35 | 17.35 | 160.90 | 159.73 | 12.15 |
| Control | 29.47 | 17.35 | 147.03 | | |
| Control | 29.48 | 17.01 | 171.25 | | |
| Vasopressin 1 μM | 30.89 | 16.31 | 584.07 | 613.64 | 228.15 |
| Vasopressin 1 μM | 32.38 | 16.23 | 401.71 | | |
| Vasopressin 1 μM | 31.14 | 16.55 | 855.13 | | |

FIG. 2: Relative expression of the hypertrophy marker MYHCB in H9C2 rat cells after stimulation by vasopressin±PDE2A inhibitor BAY 60-7550.

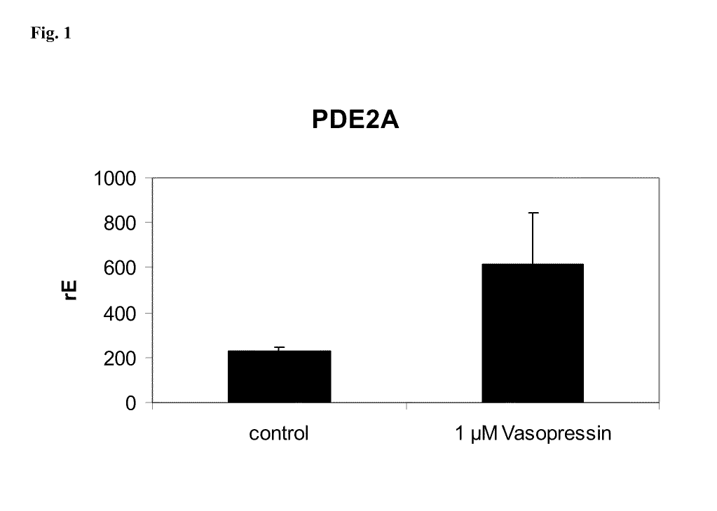

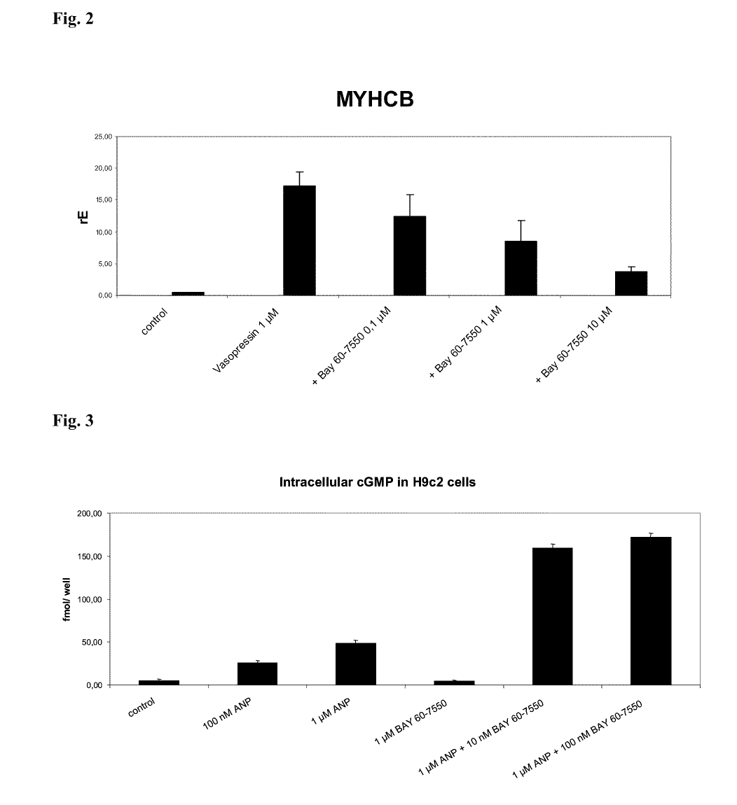

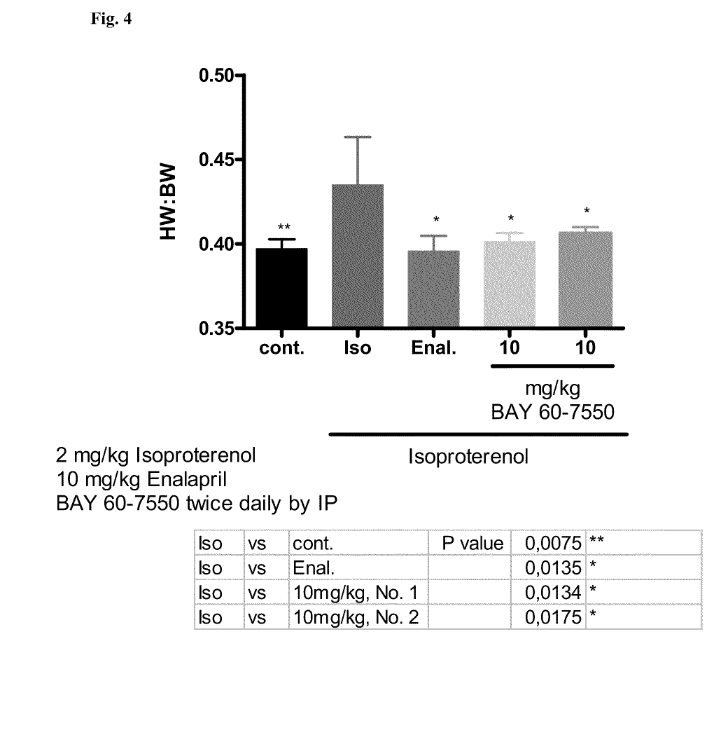

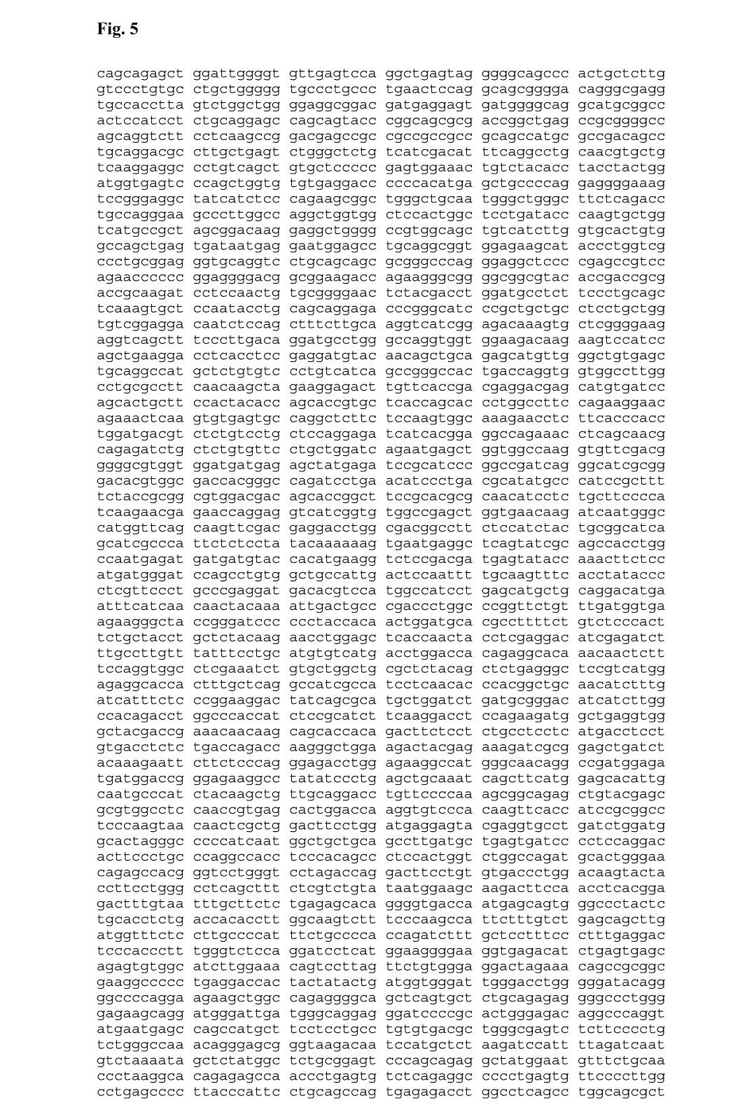

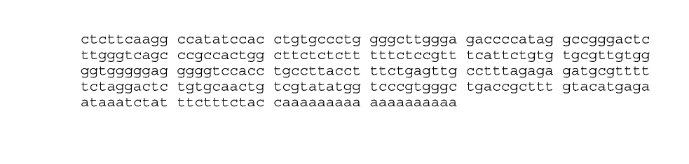

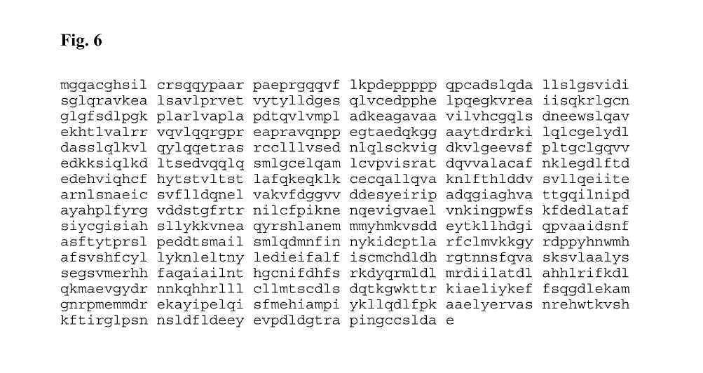

The expression of ANP and MYHCB relative to L32 ribosomal protein in H9C2 cells after incubation with vasopressin (0.1 micromol, 1 micromal, 10 micromol) for 72 h is shown. It emerges that the simultaneous presence of the PDE2A inhibitor BAY 60-7550 dose-dependently suppresses the induction of the hypertrophy marker MYHCB by vasopressin. The results are listed in a table below. (Mean relative expression rE from a duplicate determination).

| | Ct r-MYHCB | Ct r-L32 | rE | MW | STABW |
|---|---|---|---|---|---|
| Control | 34.99 | 16.07 | 0.53 | | |
| Control | 34.77 | 15.69 | 0.47 | 0.50 | 0.03 |
| Vasopressin 1 μM | 30.01 | 16.29 | 19.43 | | |
| Vasopressin 1 μM | 30.21 | 16.12 | 15.03 | 17.23 | 2.20 |
| Vasopressin 1 μM + 0.1 μM BAY 60-7550 | 31.97 | 17.14 | 9.00 | | |
| Vasopressin 1 μM + 0.1 μM BAY 60-7550 | 30.46 | 16.45 | 15.89 | 12.44 | 3.44 |
| Vasopressin 1 μM + 1 μM BAY 60-7550 | 32.76 | 17.16 | 5.28 | | |
| Vasopressin 1 μM + 1 μM BAY 60-7550 | 31.12 | 16.68 | 11.79 | 8.54 | 3.26 |
| Vasopressin 1 μM + 10 μM BAY 60-7550 | 32.72 | 16.91 | 4.56 | | |
| Vasopressin 1 μM + 10 μM BAY 60-7550 | 33.75 | 17.34 | 3.01 | 3.79 | 0.78 |

FIG. 3: Change in the intracellular cGMP concentration in H9c2 rat cardiomyocytes after stimulation with ANP in the presence of various concentrations of the PDE2A inhibitor BAY 60-7550. The cGMP content in the cells after incubation with ANP and the stated dosages of the PDE2A inhibitor BAY 60-7550 for 15 min is shown. It emerges that the PDE2A inhibitor BAY 60-7550 dose-dependently and synergistically increases the intracellular cGMP level in the H9c2 cells.

FIG. 4: Alteration in the heart weight compared with the body weight (HW:BW) by subcutaneous administration of isoprenaline (2 mg/kg/d) and the influence of enalapril as positive control 810 mg/kg/d in the drinking water) and of the PDE2A inhibitor BAY 60-7550. The heart weight:body weight ratio as a function of the treatment is shown. It emerges that in two independent groups of animals the PDE2A inhibitor BAY 60-7550 given at 10 mg/kg/d (i.p.) for 5 days is able to suppress the isoprenaline-induced increase in heart weight almost as well as the positive control enalapril.

FIG. 5: FIG. 5 shows the cDNA sequence of human PDE2A (Accession No. NM_{—}002599, SEQ ID NO: 1).

FIG. 6: FIG. 6 shows the amino acid sequence of human PDE2A (Accession No. NP_{—}002590, SEQ ID NO:2).

### INVESTIGATIONS OF PDE2A EXPRESSION AND MYHCB EXPRESSION IN H9C2 RAT CELLS

The relative expression of PDE2A in H9c2 rat cells is measured by quantifying the mRNA using the real-time polymerase chain reaction [7]. Compared with conventional PCR, the real-time PCR has the advantage of more accurate quantification by introducing an additional, fluorescence-labeled oligonucleotide. This so-called probe contains at the 5′ end the fluorescent dye FAM (6-carboy-fluorescein) and at the 3′ end the fluorescence quencher TAMRA (6-carboxy-tetramethylrhodamine). During the polymerase chain reaction, the fluorescent dye FAM is cleaved off the probe by the 5′-exonuclease activity of the Taq polymerase in the TaqMan PCR, and thus the previously quenched fluorescence signal is obtained. The number of the cycle at which the fluorescence intensity is about 10 standard deviations above the background fluorescence is recorded as the so-called threshold [treshold cyle (Ct value)].

Total RNA is isolated from the H9c2 rat cardiomyocytes from a 6-well (about 4×10⁵ cells) using an RNeasy Kit (Qiagen Hilden). 1 μg portions of total RNA per tissue are reacted with 1 unit of DNase I (from Invitrogen) at room temperature for 15 min to remove contamination by genomic DNA. The Dnase I is inactivated by adding 1 μl of EDTA (25 mM) and subsequent heating at 65° C. (10 min).

Subsequently, cDNA synthesis is carried out in the same reaction mixture in accordance with the instruction for the “SUPERSCRIPT-II RT cDNA synthesis kit” (from Invitrogen), and the reaction volume is made up to 200 μl with distilled water. For the PCR, 7.5 μl of primer and probe mixture and 12.5 μl of TaqMan reaction solution (qPCR Mastermix, from Eurogentec) are added to 5 μl portions of the diluted cDNA solution. The final concentration of the primers is 300 nM in each case, and that of the probe is 150 nM. The sequence of the forward and reverse primer for the rat PDE2A is: 5′-CCAAATCAGGGACCTCATATTCC-3′ (SEQ ID NO: 3) and 5′-GGTGTCCCACAAGTTCACCAT-3′ (SEQ ID NO: 4), and the sequence of the fluorescent probe is 5′-6FAM-AACAACTCGCTGGATTTCCTGGA-TAMRA-3′ (SEQ ID NO: 5). The sequence of the forward and reverse primer for r-MYHCB is: 5′-TGGAGAACGACAAGCAGCAG-3′ (SEQ ID NO: 6) and 5′-CCTGGCGTTGAGTGCATTTA-3′ (SEQ ID NO: 7), and the sequence of the fluorescent probe is 5′-6FAM-TGGATGAGCGACTCAAAAAGAAGGACTTTG-TAMRA-3′ (SEQ ID NO: 8).

The PCR takes place on an ABI Prism SDS-7700 apparatus (from Applied Biosystems) in accordance with the manufacturer's instructions. 40 cycles are carried out in this case. The Ct (see above) which is obtained for the respective gene in the relevant cDNA corresponds to the cycle in which the fluorescence intensity of the liberated probe is about 10 standard deviations above the background signal. A lower Ct value thus means an earlier start of amplification, i.e. the original sample contains more mRNA. To compensate for any variations in the cDNA synthesis, the expression of a so-called “housekeeping gene”, which should always be expressed to the same extent irrespective of the treatment of the cells, is also analyzed in all the investigated samples. L32 ribosomal protein is used to standardize PDE2A expression in H9c2 cells. The sequence of the forward and reverse primer for rat L32 is 5′-GAAAGAGCAGCACAGCTGGC-3′ (SEQ ID NO: 9), and 5′-TCATTCTCTTCGCTGCGTAGC-3′ (SEQ ID NO: 10), and the sequence of the probe is 5′-6FAM-TCAGAGTCACCAATCCCAACGCCA-TAMRA-3′ (SEQ ID NO: 11). The data are analyzed in the following way: the dCt value is calculated for each RNA. The dCt value is the difference between the Ct values for the candidate gene (i.e.: MYHCB or PDE2A) and the Ct value of the housekeeping gene in the respective tissue. A relative expression rE is calculated from this value by the following formula:

rE=2^{(18-dCt)}.

Determination of the cGMP Content in H9c2 Cells after Preincubation with the PDE2A Inhibitor BAY 60-7550

The intracellular cGMP content in H9c2 cells was determined with the Biotrak (EIA) Immunoassay from Amersham (catalog No. RPN 226) in accordance with the manufacturer's protocol. For this purpose, 105H9c2 cells/well are seeded in 12-well plates overnight and, after washing with 1×PBS (1 ml), are incubated with 800 μl of medium without FCS and the stated concentrations of the PDE2A inhibitor BAY 60-7550 and ANP at room temperature for 15 min. The supernatants were discarded, and the cells were mixed with 500 ml of ice-cold 70% strength ethanol. After shaking at room temperature (150 rpm) for 2 min, the plates are frozen at −20° C. overnight and, after thawing, the lysed cells are transferred into Eppendorf vessels. After evaporation of the ethanol in a speed-vac (3 h at 35° C.), the samples are reconstituted in 200 μl of assay buffer and worked up as indicated in the kit description. The fluorescence is measured at 450/570 nm in a Tecan Spectrafluor photometer. The resulting OD values are converted into fmol/well in accordance with the kit instructions on the basis of the standard calibration plot.

### PDE2A Inhibition Assay

PDE2A assay formats for identifying PDE2A inhibitors are known to the skilled worker. One example of a possible PDE2A activity assay system format is described below.

Human PDE2A (GenBank/EMBL Accession Number: NM_{—}002599, Rosman et al. Gene 1997 191, 89-95) is expressed in Sf9 insect cells with the aid of the Bac-to-Bac™ baculovirus expression system. 48 h after the infection, the cells are harvested and suspended in lysis buffer (20 mL/IL of culture, 50 mM Tris-HCl, pH 7.4, 50 mM NaCl, 1 mM MgCl2, 1.5 mM EDTA, 10% glycerol, 20 μL of Protease Inhibitor Cocktail Set III [CalBiochem, La Jolla, Calif. USA]). The cells are disrupted with the aid of ultrasound at 4° C. and then centrifuged at 15,000×g at 4° C. for 30 minutes. The supernatant (PDE2A preparation) was collected and stored at −80° C.

The test substances are dissolved and serially diluted in 100% DMSO to determine their in vitro effect on PDE2A. Serial dilutions from 200 μM to 1.6 μM are typically prepared (resulting final concentrations in the assay: 4 μM to 0.032 μM). 2 μL portions of the diluted substance solutions are placed in the wells of microtiter plates (Isoplate; Wallac Inc., Atlanta, Ga.). Then 50 μL of a dilution of the PDE2A preparation described above are added. The dilution of the PDE2A preparation is chosen so that less than 70% of the substrate is converted during the later incubation (typical dilution: 1:200 000; dilution buffer: 50 mM Tris/HCl pH 7.5; 8.3 mM MgCl2; 1.7 mM EDTA, 0.2% BSA). The substrate, [5′,8-3H] adenosine 3′,5′-cyclic phosphate (1 μCi/μL; Amersham Pharmacia Biotech., Piscataway, N.J.), is diluted 1:2000 with assay buffer (50 mM Tris/HCl pH 7.5; 8.3 mM MgCl2; 1.7 mM EDTA) to a concentration of 0.0005 μCi/μL, and cGMP (1 μM final concentration in the assay), which serves to stimulate PDE2, is added. The enzyme reaction is finally started by adding 50 μL (0.025 μCi) of this substrate solution. The assay mixtures are incubated at room temperature for 60 min, and the reaction is stopped by adding 25 μL of a suspension with 18 mg/ml of Yttrium Scintillation Proximity Beads (Amersham Pharmacia Biotech., Piscataway, N.J.). The microtiter plates are sealed with a film and left to stand at room temperature for 60 min. The plates are then measured in a Microbeta scintillation counter (Wallac Inc., Atlanta, Ga.) for 30 s per well. The IC₅₀ values are determined using a graph with a substance concentration plotted against the percentage inhibition.

### Inhibition of PDEs 1, 3, 4, 5, 7, 8, 9, 10 and 11

Recombinant human PDE3B (GenBank/EMBL Accession Number: NM_{—}000922, Miki et al. Genomics 1996 36, 476-485), PDE4B (GenBank/EMBL Accession Number: NM_{—}002600, Obernolte et al. Gene. 1993 129, 239-247), PDE7B (GenBank/EMBL Accession Number: NM_{—}018945, Hetman et al. Proc. Natl. Acad. Sci. U.S.A. 2000 97, 472-476), PDE8A (GenBank/EMBL Accession Number: AF_{—}056490, Fisher et al. Biochem. Biophys. Res. Commun. 1998 246, 570-577), PDE9A (GenBank/EMBL Accession Number: NM_{—}002606, Fisher et al. J. Biol. Chem. 1998 273, 15559-15564), PDE10A (GenBank/EMBL Accession Number: NM 06661, Fujishige et al. J. Biol. Chem. 1999 274, 18438-45, PDE11A (GenBank/EMBL Accession Number: NM_{—}016953, Fawcett et al. Proc. Natl. Acad. Sci. 2000 97, 3702-3707) were expressed in Sf9 cells with the aid of the pFASTBAC baculovirus expression system (GibcoBRL). Bovine PDE1 was purchased from Sigma-Aldrich (P 9529). PDE5 was removed from human blood platelets by ultrasound treatment followed by a centrifugation and column chromatography of the supernatant on Mono Q 10/10 (linear NaCl gradient, elution with 0.2-0.3 M NaCl in 20 mM Hepes pH 7.2, 2 mM MgCl2).

The in vitro effect of test substances on recombinant PDE3B, PDE4B, PDE7B, PDE8A, PDE10A and PDE11A is determined by the assay protocol described above for PDE2A, where there is no addition to the assay of the cGMP used to stimulate PDE2A. To determine a corresponding effect on PDE1, PDE5 and PDE9A, the protocol is additionally modified as follows: for PDE1 additionally Calmodulin 10⁻⁷ M and CaCl2 3 mM are added to the reaction mixture. For PDE5 and PDE9A, the substrate used is [8-3H] cGMP (1 μCi/μL; Amersham Pharmacia Biotech., Piscataway, N.J.) in the dilution mentioned above. In order to stop the PDE9A reaction, 25 μl of a PDE9A inhibitor C e.g. BAY 73-6691, 5 μM of final concentration) dissolved in assay buffer are added immediately before addition of the Yttrium Scintillation Proximity Bead suspension.

PDE2A inhibitors may also act at the level of transcription or translation of PDE2A. Assay systems for finding corresponding inhibitors are well known to the skilled worker.

Assay of PDE2A inhibitors for anti-hypertrophic effect in vivo:

The antihypertrophic effect of the PDE2A inhibitor BAY 60-7550 is assayed by using the so-called mouse isoprenaline model [ ]. This involves 8 mice (C57b1/6 strain) per dose group receiving subcutaneous administration of isoprenaline at a dose of 2 mg/kg/d for 5 days, while the control group receives a saline solution as vehicle control. As positive control, in addition to the isoprenaline, the ACE inhibitor enalapril was administered in a dose of 10 mg/kg/d via the drinking water to one group, while two further groups received in addition to the isoprenaline the PDE2A inhibitor BAY 60-7550 administered intraperitoneally with a dose of 10 mg/kg/d. As a measure of the degree of cardiac hypertrophy, the heart weight/body weight (HW:BW) ratio is determined after 5 d, and the relation of the effect of the substances to the effect of isoprenaline is found.

### PDE2A Inhibitor Formulations

The PDE2A inhibitors can be converted in a known manner into the usual formulations such as tablets, coated tablets, pills, granules, aerosols, syrups, emulsions, suspensions and solutions, by using inert, non-toxic, pharmaceutically suitable carriers or solvents. In this case, the therapeutically effective compound is to be present in each case in a concentration of from 0.5 to 90% by weight of the complete mixture, i.e. in amounts which suffice to reach the stated dosage range.

The formulations are produced for example by extending the active ingredients with solvents and/or carriers, where appropriate with use of emulsifiers and/or dispersants, it being possible for example in the case where water is used as diluent where appropriate to use organic solvents as auxiliary solvents.

Administration takes place in the usual way, preferably orally, transdermally, intravenously or parenterally, especially orally or intravenously. However, it can also take place by inhalation through the mouth or the nose, for example with the aid of a spray, or topically via the skin.

It has generally proved advantageous to administer amounts of something 0.001 to 10 mg/kg, on oral use preferably about 0.005 to 3 mg/kg, of body weight to achieve effective results. It may nevertheless be necessary, where appropriate, to deviate from the stated amounts, in particular as a function of body weight or the nature of the administration route, the individual response to the medicament, the type of formulation thereof and the time or interval over which administration takes place. Thus, in some cases it may be sufficient to make do with less than the aforementioned minimum amount, whereas in other cases the upper limit mentioned must be exceeded. Where relatively large amounts are administered, it may be advisable to distribute these in a plurality of single doses over the day.

### LITERATURE

1. Scherer, C R, Dissertation Univ. Frankfurt, 2002.
2. Brostrom M A, Reilly B A, Wilson F J, Brostrom C O. Vasopressin-induced hypertrophy in H9c2 heart-derived myocytes. Int J Biochem Cell Biol. 2000 September; 32(9):993-1006.
3. Calderone A, Thaik C M, Takahashi N, Chang D L, Colucci M., Nitric oxide, atrial natriuretic peptide, and cyclic GMP inhibit the growth-promoting effects of norepinephrine in cardiac myocytes and fibroblasts. J. Clin Invest. 1998 Feb. 15; 101(4):812-8.
4. Booz G W, Putting the brakes on cardiac hypertrophy: exploiting the NO-cGMP counter-regulatory system. Hypertension. 2005 March; 45(3):341-6.
5. Mendelsohn M E, Nat. Med. 11, 2005, 115-116
6. Boess F G, Hendrix M, van der Staay F J, Erb C, Schreiber R, van Staveren W, de Vente J, Prickaerts J, Blokland A, Koenig G. Inhibition of phosphodiesterase 2 increases neuronal cGMP, synaptic plasticity and memory performance, Neuropharmacology. 2004 December; 47(7): 1081-92
7. Heid C A, Stevens J, Livak K J, Williams P M., Real time quantitative PCR. Genome Res 6 (1996), 986-994.
8. Hassan M A, Ketat A F., Sildenafil citrate increases myocardial cGMP content in rat heart, decreases its hypertrophic response to isoproterenol and decreases myocardial leak of creatine kinase and troponin T, BMC Pharmacol. 2005 Apr. 6; 5(1): 10.

### REFERENCES

ANP Atrial natriuretic peptide
AVP: Arginine-vasopressin
BW: Body weight
Ct: Threshold cycle
HW: Heart weight
MYHCB: Myosin heavy chain beta subunit
PBS Phosphate-buffered saline
rE: Relative expression
SD: Standard deviation

1-13. (canceled)

14. A method of treating a disorder selected from the group consisting of heart failure, a cardiomyopathy underlying heart failure, coronary heart diseases, stable and unstable angina pectoris, acute myocardial infarction, sudden heart death, high blood pressure, a sequela of atherosclerosis, a vascular disorder, a disorder of the kidney, and erectile dysfunction, comprising administering to a patient in need thereof a pharmaceutically effective amount of a compound of the general formula (I), 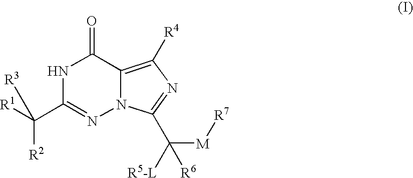
in which
R¹ is phenyl, naphthyl, quinolinyl or isoquinolinyl, each of which may be substituted up to three times, identically or differently, by radicals selected from the group consisting of (C₁-C₄)-alkyl, (C₁-C₄)-alkoxy, halogen, cyano, —NHCOR⁸, —NHSO₂R⁹, —SO₂NR¹⁰R¹¹, —SO₂R¹², and —NR¹³R¹⁴,
in which R⁸, R¹⁰, R¹¹, R¹³ and R¹⁴ are independently of one another hydrogen or (C₁-C₄)-alkyl, and R⁹ and R¹² are independently of one another (C₁-C₄)-alkyl,
or
R¹⁰ and R¹¹ together with the adjacent nitrogen atom form an azetidin-1-yl, pyrrol-1-yl, piperid-1-yl, azepin-1-yl, 4-methylpiperazin-1-yl or morpholin-1-yl radical,
or
R¹³ and R¹⁴ together with the adjacent nitrogen atom form an azetidin-1-yl, pyrrol-1-yl, piperid-1-yl, azepin-1-yl, 4-methylpiperazin-1-yl or morpholin-1-yl radical,
R² and R³ are independently of one another hydrogen or fluorine,
R⁴ is (C₁-C₄)-alkyl,
R⁵ is (C₁-C₃)-alkyl,
R⁶ is hydrogen or methyl,
R⁷ is phenyl, thiophenyl, furanyl, each of which may be substituted up to three times identically or differently by radicals selected from the group consisting of (C₁-C₄)-alkyl, (C₁-C₄)-alkoxy, halogen and cyano, or is (C₅-C₈)-cycloalkyl,
L is carbonyl or hydroxymethanediyl, and
M is (C₂-C₅)-alkanediyl, (C₂-C₅)-alkenediyl or (C₂-C₅)-alkynediyl,
and the physiologically tolerated salts thereof.

15. The method of claim 14 wherein R¹ is phenyl whose meta and/or para positions are substituted up to three times identically or differently by radicals selected from the group consisting of (C₁-C₄)-alkyl, (C₁-C₄)-alkoxy and —SO₂NR¹⁰R¹¹, and in which R¹⁰ and R¹¹ have the meaning indicated in claim 14.

16. The method of claim 14, wherein R⁷ is phenyl.

17. The method of claim 14, wherein
R¹ is phenyl whose meta and/or para positions are substituted up to three times identically or differently by radicals selected from the group consisting of (C₁-C₄)-alkyl, (C₁-C₄)-alkoxy and —SO₂NR¹⁰R¹¹, or naphthyl or quinolinyl,
in which R¹⁰ and R¹¹ are independently of one another hydrogen or (C₁-C₄)-alkyl,
R² and R³ are hydrogen,
R⁴ is methyl or ethyl,
R⁵ is methyl,
R⁶ is hydrogen or methyl,
L is carbonyl or hydroxymethanediyl, and
M is straight-chain (C₂-C₅)-alkane-1,ω-diyl, straight-chain (C₂-C₅)-alkene-1,ω-diyl or straight-chain (C₂-C₅)-alkyne-1,ω-diyl.

18. A method of treating a disorder selected from the group consisting of heart failure, a cardiomyopathy underlying heart failure, coronary heart diseases, stable and unstable angina pectoris, acute myocardial infarction, sudden heart death, high blood pressure, a sequela of atherosclerosis, a vascular disorder, a disorder of the kidney, and erectile dysfunction, comprising administering to a patient in need thereof a pharmaceutically effective amount of a compound of the general formula (II), 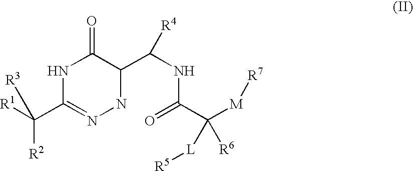
in which R¹, R², R³, R⁴, R⁵, R⁶, R⁷, L and M have the meaning indicated in claim 1, and the salts thereof.

19. A method of treating a disorder selected from the group consisting of heart failure, a cardiomyopathy underlying heart failure, coronary heart diseases, stable and unstable angina pectoris, acute myocardial infarction, sudden heart death, high blood pressure, a sequela of atherosclerosis, a vascular disorder, a disorder of the kidney, and erectile dysfunction, comprising administering to a patient in need thereof a pharmaceutically effective amount of a compound 2-(3,4-dimethoxybenzyl)-7-[1-(1-hydroxyethyl)-4-phenylbutyl]-5-methylimidazo[5,1f][1,2,4]triazin-4(3H)-one having the structural formula:

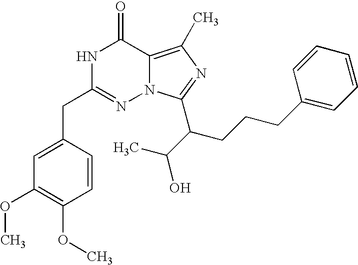

20. The method of claim 14 wherein, the heart failure is a heart failure induced by a cardiomyopathy selected from the group of cardiomyopathies consisting of dilated cardiomyopathy (DCM), restrictive cardiomyopathy (RCM), arrhythmogenic right-ventricular cardiomyopathy (ARVCM), myocarditis and/or hypertrophic cardiomyopathy (HCM).


SEQUENCE LISTING (11 sequences)

SEQ ID NO: 1 - DNA, 4240 bp, Homo sapiens
cagcagagctggattggggtgttgagtccaggctgagtagggggcagcccactgctcttggtccctgtgcctgctgggggtgccctgccctgaactccaggcagcggggacagggcgaggtgccaccttagtctggctggggaggcggacgatgaggagtgatggggcaggcatgcggccactccatcctctgcaggagccagcagtacccggcagcgcgaccggctgagccgcggggccagcaggtcttcctcaagccggacgagccgccgccgccgccgcagccatgcgccgacagcctgcaggacgccttgctgagtctgggctctgtcatcgacatttcaggcctgcaacgtgctgtcaaggaggccctgtcagctgtgctcccccgagtggaaactgtctacacctacctactggatggtgagtcccagctggtgtgtgaggaccccccacatgagctgccccaggaggggaaagtccgggaggctatcatctcccagaagcggctgggctgcaatgggctgggcttctcagacctgccagggaagcccttggccaggctggtggctccactggctcctgatacccaagtgctggtcatgccgctagcggacaaggaggctggggccgtggcagctgtcatcttggtgcactgtggccagctgagtgataatgaggaatggagcctgcaggcggtggagaagcataccctggtcgccctgcggagggtgcaggtcctgcagcagcgcgggcccagggaggctccccgagccgtccagaaccccccggaggggacggcggaagaccagaagggcggggcggcgtacaccgaccgcgaccgcaagatcctccaactgtgcggggaactctacgacctggatgcctcttccctgcagctcaaagtgctccaatacctgcagcaggagacccgggcatcccgctgctgcctcctgctggtgtcggaggacaatctccagctttcttgcaaggtcatcggagacaaagtgctcggggaagaggtcagctttcccttgacaggatgcctgggccaggtggtggaagacaagaagtccatccagctgaaggacctcacctccgaggatgtacaacagctgcagagcatgttgggctgtgagctgcaggccatgctctgtgtccctgtcatcagccgggccactgaccaggtggtggccttggcctgcgccttcaacaagctagaaggagacttgttcaccgacgaggacgagcatgtgatccagcactgcttccactacaccagcaccgtgctcaccagcaccctggccttccagaaggaacagaaactcaagtgtgagtgccaggctcttctccaagtggcaaagaacctcttcacccacctggatgacgtctctgtcctgctccaggagatcatcacggaggccagaaacctcagcaacgcagagatctgctctgtgttcctgctggatcagaatgagctggtggccaaggtgttcgacgggggcgtggtggatgatgagagctatgagatccgcatcccggccgatcagggcatcgcgggacacgtggcgaccacgggccagatcctgaacatccctgacgcatatgcccatccgcttttctaccgcggcgtggacgacagcaccggcttccgcacgcgcaacatcctctgcttccccatcaagaacgagaaccaggaggtcatcggtgtggccgagctggtgaacaagatcaatgggccatggttcagcaagttcgacgaggacctggcgacggccttctccatctactgcggcatcagcatcgcccattctctcctatacaaaaaagtgaatgaggctcagtatcgcagccacctggccaatgagatgatgatgtaccacatgaaggtctccgacgatgagtataccaaacttctccatgatgggatccagcctgtggctgccattgactccaattttgcaagtttcacctatacccctcgttccctgcccgaggatgacacgtccatggccatcctgagcatgctgcaggacatgaatttcatcaacaactacaaaattgactgcccgaccctggcccggttctgtttgatggtgaagaagggctaccgggatcccccctaccacaactggatgcacgccttttctgtctcccacttctgctacctgctctacaagaacctggagctcaccaactacctcgaggacatcgagatctttgccttgtttatttcctgcatgtgtcatgacctggaccacagaggcacaaacaactctttccaggtggcctcgaaatctgtgctggctgcgctctacagctctgagggctccgtcatggagaggcaccactttgctcaggccatcgccatcctcaacacccacggctgcaacatctttgatcatttctcccggaaggactatcagcgcatgctggatctgatgcgggacatcatcttggccacagacctggcccaccatctccgcatcttcaaggacctccagaagatggctgaggtgggctacgaccgaaacaacaagcagcaccacagacttctcctctgcctcctcatgacctcctgtgacctctctgaccagaccaagggctggaagactacgagaaagatcgcggagctgatctacaaagaattcttctcccagggagacctggagaaggccatgggcaacaggccgatggagatgatggaccgggagaaggcctatatccctgagctgcaaatcagcttcatggagcacattgcaatgcccatctacaagctgttgcaggacctgttccccaaagcggcagagctgtacgagcgcgtggcctccaaccgtgagcactggaccaaggtgtcccacaagttcaccatccgcggcctcccaagtaacaactcgctggacttcctggatgaggagtacgaggtgcctgatctggatggcactagggcccccatcaatggctgctgcagccttgatgctgagtgatcccctccaggacacttccctgcccaggccacctcccacagccctccactggtctggccagatgcactgggaacagagccacgggtcctgggtcctagaccaggacttcctgtgtgaccctggacaagtactaccttcctgggcctcagctttctcgtctgtataatggaagcaagacttccaacctcacggagactttgtaatttgcttctctgagagcacaggggtgaccaatgagcagtgggccctactctgcacctctgaccacaccttggcaagtctttcccaagccattctttgtctgagcagcttgatggtttctccttgccccatttctgccccaccagatctttgctcctttccctttgaggactcccaccctttgggtctccaggatcctcatggaaggggaaggtgagacatctgagtgagcagagtgtggcatcttggaaacagtccttagttctgtgggaggactagaaacagccgcggcgaaggccccctgaggaccactactatactgatggtgggattgggacctgggggatacaggggccccaggaagaagctggccagaggggcagctcagtgctctgcagagaggggccctggggagaagcaggatgggattgatgggcaggagggatccccgcactgggagacaggcccaggtatgaatgagccagccatgcttcctcctgcctgtgtgacgctgggcgagtctcttcccctgtctgggccaaacagggagcgggtaagacaatccatgctctaagatccattttagatcaatgtctaaaatagctctatggctctgcggagtcccagcagaggctatggaatgtttctgcaaccctaaggcacagagagccaaccctgagtgtctcagaggccccctgagtgttccccttggcctgagccccttacccattcctgcagccagtgagagacctggcctcagcctggcagcgctctcttcaaggccatatccacctgtgccctggggcttgggagaccccataggccgggactcttgggtcagcccgccactggcttctctctttttctccgtttcattctgtgtgcgttgtggggtgggggagggggtccacctgccttacctttctgagttgcctttagagagatgcgtttttctaggactctgtgcaactgtcgtatatggtcccgtgggctgaccgctttgtacatgagaataaatctatttctttctaccaaaaaaaaaaaaaaaaaaa

SEQ ID NO: 2 - protein, 941 aa, Homo sapiens
MGQACGHSILCRSQQYPAARPAEPRGQQVFLKPDEPPPPPQPCADSLQDALLSLGSVIDISGLQRAVKEALSAVLPRVETVYTYLLDGESQLVCEDPPHELPQEGKVREAIISQKRLGCNGLGFSDLPGKPLARLVAPLAPDTQVLVMPLADKEAGAVAAVILVHCGQLSDNEEWSLQAVEKHTLVALRRVQVLQQRGPREAPRAVQNPPEGTAEDQKGGAAYTDRDRKILQLCGELYDLDASSLQLKVLQYLQQETRASRCCLLLVSEDNLQLSCKVIGDKVLGEEVSFPLTGCLGQVVEDKKSIQLKDLTSEDVQQLQSMLGCELQAMLCVPVISRATDQVVALACAFNKLEGDLFTDEDEHVIQHCFHYTSTVLTSTLAFQKEQKLKCECQALLQVAKNLFTHLDDVSVLLQEIITEARNLSNAEICSVFLLDQNELVAKVFDGGVVDDESYEIRIPADQGIAGHVATTGQILNIPDAYAHPLFYRGVDDSTGFRTRNILCFPIKNENQEVIGVAELVNKINGPWFSKFDEDLATAFSIYCGISIAHSLLYKKVNEAQYRSHLANEMMMYHMKVSDDEYTKLLHDGIQPVAAIDSNFASFTYTPRSLPEDDTSMAILSMLQDMNFINNYKIDCPTLARFCLMVKKGYRDPPYHNWMHAFSVSHFCYLLYKNLELTNYLEDIEIFALFISCMCHDLDHRGTNNSFQVASKSVLAALYSSEGSVMERHHFAQAIAILNTHGCNIFDHFSRKDYQRMLDLMRDIILATDLAHHLRIFKDLQKMAEVGYDRNNKQHHRLLLCLLMTSCDLSDQTKGWKTTRKIAELIYKEFFSQGDLEKAMGNRPMEMMDREKAYIPELQISFMEHIAMPIYKLLQDLFPKAAELYERVASNREHWTKVSHKFTIRGLPSNNSLDFLDEEYEVPDLDGTRAPINGCCSLDAE

SEQ ID NO: 3 - DNA, 23 bp, artificial sequence
  primer
ccaaatcagggacctcatattcc

SEQ ID NO: 4 - DNA, 21 bp, artificial sequence
  primer
ggtgtcccacaagttcaccat

SEQ ID NO: 5 - DNA, 23 bp, artificial sequence
  probe
aacaactcgctggatttcctgga

SEQ ID NO: 6 - DNA, 20 bp, artificial sequence
  primer
tggagaacgacaagcagcag

SEQ ID NO: 7 - DNA, 20 bp, artificial sequence
  primer
cctggcgttgagtgcattta

SEQ ID NO: 8 - DNA, 30 bp, artificial sequence
  probe
tggatgagcgactcaaaaagaaggactttg

SEQ ID NO: 9 - DNA, 20 bp, artificial sequence
  primer
gaaagagcagcacagctggc

SEQ ID NO: 10 - DNA, 21 bp, artificial sequence
  primer
tcattctcttcgctgcgtagc

SEQ ID NO: 11 - DNA, 24 bp, artificial sequence
  probe
tcagagtcaccaatcccaacgcca